(19) United States
(12) Patent Application Publication
(10) Pub. No.: US 2007/0066543 A1
(43) Pub. Date: Mar. 22, 2007

(54) Treatment of tay sachs or sandhoff diseases by enhancing hexosaminidase activity

(75) Inventors: Don Mahuran, Toronto (CA); Michael Tropak, Toronto (CA); Stephen Withers, Vancouver (CA)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignees: The Hospital For Sick Children, Toronto (CA); The University Of British Columbia, Vancouver (CA)

(21) Appl. No.: 10/557,454

(22) PCT Filed: May 21, 2004
(86) PCT No.: PCT/CA04/00758

§ 371 Date: Jan. 26, 2006

Related U.S. Application Data
(60) Provisional application No. 60/472,439, filed on May 22, 2003.

(51) Int. Cl. A61K 31/429; A61K 31/70

(52) U.S. Cl. 514/23; 514/367

(57) ABSTRACT

The invention provides a method for treating an animal suffering from a disease associated with reduced activity of a lysosomal hexosaminidase by administering to the animal an effective amount of a compound which increases the activity of the hexosaminidase.

### FIELD OF THE INVENTION

The invention relates to methods and compositions for treating genetic diseases, and, more particularly, to methods and compositions for treating diseases associated with reduced activity of lysosomal hexosaminidases.

### BACKGROUND OF THE INVENTION

Lysosomal Storage Diseases (LSD) are a group of genetic diseases in which inadequate levels of a catabolic enzyme in the lysosome results in damaging intralysosomal accumulation of various substrates. Clinical symptoms appear when the activity of the mutant lysosomal enzyme is reduced below a “critical threshold”. Surprisingly, the critical threshold is quite low, as asymptomatic individuals have been identified with enzyme activity levels of 10-20% of wild type (WT), whereas symptomatic patients have levels of 0-5% of WT. Because as little as 10% WT enzyme activity is sufficient for individuals to be asymptomatic, successful pharmacological treatment of these diseases would require only a modest increase in the residual enzyme activity.

Many of the mutations responsible for LSD do not directly affect enzyme activity, but instead result in a misfolded protein. A large percentage of mutated and a small percentage of WT proteins are unable to reach their final conformation, necessary for transport out of the endoplasmic reticulum (ER) and into the lysosome. These are recognized by the ER quality control system and undergo proteolytic degradation. Pharmacological chaperones are small chemical compounds which specifically bind to a target protein and stabilize its native conformation. By directly augmenting the folding efficiency of a protein, these compounds increase the amount of functional mutant and WT protein targeted to the lysosome. Sub-inhibitory concentrations of active-site inhibitors of α-galactosidase, a monomeric lysosomal enzyme, have been shown to act in this manner, alleviating symptoms of Fabry disease in affected mice and humans.

Lysosomal β-N-acetyl-hexosaminidases catalyse the hydrolysis of terminal neutral or negatively charged N-acetyl galactosamines and glucosamines at the glycosidic linkage from oligosaccharides or glycolipids. β-hexosaminidase exists as one of three possible dimers, resulting from the combinatorial assembly of an alpha subunit and/or beta subunit. Whereas the physiologically relevant dimers hexosaminidase A (α and β subunit) and hexosaminidase B (two β subunits) can hydrolyse glycolipids and oligosaccharides terminating with a neutral N-acetyl hexosamine, only hexosaminidase A can utilize sialic acid-containing GM₂ ganglioside as substrate.

Hydrolysis of terminal N-acetyl galactosamine from a negatively charged glycolipid or GM2 ganglioside also requires association of hexosaminidase A with the GM2 activator protein, which facilitates the removal of GM₂ from its membranous environment and presents the glycolipid head group to hexosaminidase A. A molecular model of the active complex has recently been published. Mutations resulting in a complete absence of hexosaminidase A, or in an impaired protein, disturb the ganglioside catabolic pathway, resulting in variable accumulation of GM2/GA2.

Tay-Sachs Disease (TSD) is a lysosomal storage disease associated with mutations in the gene encoding the a subunit of lysosomal β-hexosaminidase A. The infantile form of Tay-Sachs, the severest form of the disease, is associated with mutations which result in the production of little or no protein, with undetectable hexosaminidase A activity. Without hexosaminidase A activity, the GM2 ganglioside accumulates in cells, particularly in the brain, leading to progressive neurological damage and death in early childhood. Neurons from patients with late stage Tay-Sachs disease, which have large accumulations of GM2 ganglioside, are believed to undergo apoptosis, in part accounting for the neurologic deficits seen in the disease.

The variable onset, adult form of Tay-Sachs disease (ATSD) is commonly associated with missense mutations in the α-subunit of the enzyme. Most patients with adult Tay-Sachs disease have a Gly to Ser mutation at position 269 (G269S) in the α-subunit of β-hexosaminidase A, resulting in an unstable α subunit that retains the potential of forming an active enzyme dimer.

Another lysosomal storage disease associated with mutant forms of hexosaminidase is Sandhoff disease, both adult Sandhoff disease (ASD) and infant Sandhoff disease (ISD), which are associated with mutations affecting the gene encoding the β subunit of hexosaminidases A and B.

Almost the only currently available therapeutic approach for treatment of ATSD or ASD patients is the use of n-butyl-DNJ (NB-DNJ) to inhibit the synthesis of GM2 and other higher gangliosides (Platt et al., (1997), Science, v. 276, pp. 428-431; Sango et al., (1995), Nature Genet., v.11, pp. 170-176).

The goal of this “substrate deprivation” approach is to reduce GM₂ levels to a point below the maximum turnover rate of the patient's defective hexosaminidase A activity. This approach, however, is associated with several problems. NB-DNJ is toxic to liver and spleen, oligosaccharides produced by glycoprotein degradation continue to accumulate in the lysosomes in ASD patients and the effects of the treatment are non-specific and may affect other ganglioside biosynthesis pathways.

No fully satisfactory therapies exist for any form of Tay-Sachs disease or for Sandhoff disease. There remains, therefore, an acute need for better treatments which can ameliorate these debilitating diseases.

### SUMMARY OF THE INVENTION

In accordance with one embodiment, there is provided a method for treating an animal suffering from a disease associated with reduced activity of a lysosomal hexosaminidase by administering to the animal an effective amount of a compound which increases the activity of the hexosaminidase.

In accordance with a further embodiment, there is provided a method of modulating the activity of a mammalian hexosaminidase A enzyme comprising contacting the enzyme with a compound which stabilises a subunit protein of the enzyme.

In accordance with a further embodiment, there is provided a method for identifying a candidate compound for treatment of a disease associated with reduced activity of a hexosaminidase comprising determining the ability of the compound to increase the activity of the hexosaminidase

### SUMMARY OF THE DRAWINGS

Certain embodiments of the invention are described, reference being made to the accompanying drawings, wherein:

FIG. 1A shows examples of compounds which competitively inhibit hexosaminidase A.

FIG. 1B shows the structures of NGT and NGal-T. FIG. 2 shows hexosaminidase A activity in adult Tay-Sachs disease (ATSD) cell line 17662 treated with the indicated inhibitory compounds at various concentrations.

FIG. 3A shows % remaining activity of wild type and 17662 hexosaminidase A enzyme after incubation for various time periods at 42° C.

FIG. 3B shows residual hexosaminidase A activity after incubation at 42° C. for 30′ or 60′ in the presence of various inhibitors.

FIG. 4A shows a Western blot of hexosaminidase A levels in ATSD cells cultured in the presence of various inhibitory compounds. The 60 kD band corresponds to α subunit, as shown enlarged in FIG. 4B.

FIG. 5A shows hexosaminidase A activity in wild type fibroblasts (WT), cells from adult onset Tay-Sachs (ATSD) and infantile Tay-Sachs (ITSD) treated with various concentrations of ACAS.

FIG. 5B shows hexosaminidase A activity in ATSD cells over four days in culture after removal of ACAS.

FIG. 6 shows enhancement of hexosaminidase A activity in ATSD fibroblasts by a panel of inhibitors at different concentrations. ATSD fibroblasts were grown in media containing or lacking inhibitors for five days. Cells were washed and lysed and hexosaminidase A activity was monitored by increase in fluorescence from release of Methyl umbelliferyl following hydrolysis of MUGS. Increase in hexosaminidase A activity is expressed as increase in fluorescence relative to fluorescence associated with untreated cells. Symbols corresponding to compound used to treat cells are shown in the legend to the right of the graph.

FIGS. 7A and 7C show hexosaminidase A activity of ASTD cells incubated with the indicated concentrations of NGT (FIG. 7A) or ACAS (FIG. 7C) for the indicated number of days.

FIGS. 7B and 7D show hexosaminidase A activity in the ASTD cells of FIGS. 7A and 7C at the indicated number of days after removal of NGT (FIG. 7B) or ACAS (FIG. 7D) from the growth medium.

FIG. 8 shows hexosaminidase A activity in wild type (WT), adult onset Tay-Sachs (ATSD) and infantile Tay-Sachs (ITSD) fibroblasts treated with various concentrations of ACAS.

FIG. 9A shows a Western blot of cell lysates from ASTD cells untreated (U) or treated with NGT, ACAS, AddNJ, GaINAc or DNJ, and lysates from wild type (WT) and ITSD cells, bands being identified using an anti-hexosaminidase A antibody.

FIG. 9B shows the specific activity of hexosaminidase A from ATSD cells treated with the indicated inhibitor concentrations.

FIG. 9C shows a cellulose acetate electrophoresis of purified hexosaminidase A (Hex A) and of hexosaminidase isoforms in lysates of ASTD cells untreated or treated with the indicated inhibitors.

FIG. 10A shows the specific activity of hexosaminidase A from (left to right) lysate of untreated ASTD cells, lysate of NGT-treated ASTD cells, lysosomal fraction from untreated ASTD cells and lysosomal fraction from NGT-treated ASTD cells.

FIG. 10B is a Western blot of the preparations of FIG. 10A, probed with an antibody to the ER protein calnexin.

FIG. 11 shows % remaining hexosaminidase A activity (Y axis) after various times of incubation at 42° C. (X axis) for WT enzyme and mutant ATSD enzyme.

FIG. 12 shows % remaining hexosaminidase A activity of mutant ATSD enzyme incubated at 42° C. for various times in the presence of the indicated compounds.

FIG. 13 shows hexosaminidase A levels in serum of control mice (0), or mice treated with 10 mg (1), 40 mg (2) or 100 mg (3) NGT. Each symbol represents one mouse.

FIG. 14 shows β-D-mannosidase levels in serum of the mice of FIG. 13. Each symbol represents one mouse.

FIG. 15 shows hexosaminidase A activity in fibroblast cell lines from homozygous adult onset Tay-Sachs (ATSD), heterozygous adult onset Tay-Sachs (Het ATSD), infantile Tay-Sachs (ITSD and 4917), adult Sandhoff (ASD), and infantile Sandhoff (ISD), treated with various concentrations of ACAS.

FIG. 16 shows hexosaminidase A activity in the same fibroblast cell lines as FIG. 15 treated with various concentrations of NGT.

FIG. 17A shows plasma hexosaminidase activity in mice 2 days after the indicated doses of NGT.

FIG. 17B shows the ratio of hexosaminidase A and B: hexosaminidase A activity in the plasma of the mice of FIG. 17A.

FIG. 18A shows brain hexosaminidase activity in mice treated with NGT every 4 days for 15 days and in control mice.

FIG. 18B shows the ratio of hexosaminidase A and B: hexosaminidase A activity in the brains of the mice of FIG. 18A.

FIG. 19 shows the activity of hexosaminidase B at various times at 60° C. in the presence (shaded diamond) and absence (open circle) of 2.4 μm NAG-thiazoline.

FIG. 20 shows the hexosaminidase A/S activity of lysates of ATSD cells (Panel A) and ISD cells (Panel C) in the presence of NGT (circle) or GalNAct and the acid phosphatase activity of ATSD cells (Panel B) and ISD cells (Panel D) in the presence of the same compounds.

FIG. 21 shows the hexosaminidase A/S activity (MUGS), hexosaminidase A activity (MUG) and acid phosphatase activity (MUP) of lysates of ATSD cells in the presence of various concentrations of fully acetylated NGT (Panel A) or NGT (Panel B).

### DETAILED DESCRIPTION OF THE INVENTION

As used herein, “hexosaminidase A activity” means the activity of the hexosaminidase A isozyme.

As used herein, “hexosaminidase activity” means the total activity of all hexosaminidase isozymes.

The invention provides a method for treating an animal suffering from a disease associated with reduced activity of a lysosomal hexosaminidase by administering to the animal an effective amount of a compound which increases the activity of the hexosaminidase. The animal may be a human.

The invention further provides pharmaceutical compositions for treating an animal suffering from a disease associated with reduced activity of a lysosomal hexosaminidase comprising an effective amount of a compound which increases the activity of the hexosaminidase. The composition may include a pharmaceutically acceptable carrier or vehicle.

Such diseases include the subacute or juvenile form (TSD) and the chronic or adult form of Tay-Sachs disease (ATSD) and the adult form of Sandhoff disease (ASD), which are associated with reduced activity of hexosaminidase. In Sandhoff disease, there is also a reduction in. hexosaminidase B activity, with the predominant hexosaminidase activity being associated with hexosaminidase S. Neither B nor S is believed to be of physiological importance in normal individuals.

The inventors have shown that hexosaminidase A activity can be improved in cells from adult Tay-Sachs Disease patients by administration of competitive inhibitors of the enzyme, in a sub-inhibitory amount.

The mechanism by which a missense mutation leads to low or undetectable levels of hexosaminidase A activity in lysosomes has not been fully elucidated.

Although not wishing to be bound by the following hypothesis, it is proposed that the unstable α-subunit of hexosaminidase A found in Tay-Sachs patients is recognised by the endoplasmic reticulum quality control system and undergoes proteasome-mediated proteolytic degradation. Only a minor portion of the protein attains a conformation which is competent for transport out of the endoplasmic reticulum to the lysosome. The lysosomal hexosaminidase activity therefore does not rise above a critical threshold level, resulting in abnormally high concentrations of the ganglioside GM2.

Treatments which promote the stability of the mutant α subunit of hexosaminidase A, and therefore assist it to reach its proper conformation, could provide increased hexosaminidase activity and modest increases in hexosaminidase activity are likely to be sufficient to bring a particular patient above the threshold activity level, beyond which damaging substrate accumulations do not occur or are lessened.

Active hexosaminidases A and B consist of dimers, of an α and β subunit or two β subunits respectively. The individual monomers lack catalytic activity. It might therefore be doubted that competitive inhibitors of the active enzyme would interact with the individual monomeric subunits.

The present inventors have found that compounds which competitively inhibit the activity of hexosaminidase A in vitro can lead to improved hexosaminidase A activity in cells when administered in sub-inhibitory amounts to mutant protein-containing cells from adult onset Tay-Sachs sufferers. These compounds may act as pharmacological chaperones.

Patients with subacute TSD typically have about 2 to 5% residual hexosaminidase activity and those with chronic ATSD typically have about 5 to 10% residual activity.

The increases in hexosaminidase A activity seen in ATSD cells treated in accordance with the invention, of the order of 3 to 6 fold increase, are sufficient to raise the level of activity above the threshold required for a typical chronic patient and many subacute patients to become asymptomatic.

The enhancement of hexosaminidase A activity by administration of a competitive inhibitor has been shown to be effective also in vivo in studies using normal mice. These studies also showed that NGT is able to cross the blood-brain barrier which is crucially important in a disease such as Tay-Sachs where the disease process involves neuronal cells, particularly those in the brain, which are not accessible for enzyme replace therapy.

The invention provides methods and pharmaceutical compositions for treating adult or juvenile onset Tay-Sachs disease by administering compounds which increase hexosaminidase A activity.

In addition to the use of competitive inhibitors, any compound which can improve the stability of hexosaminidase A may be employed for treatment. Such compounds will be referred to collectively as hexosaminidase enhancers.

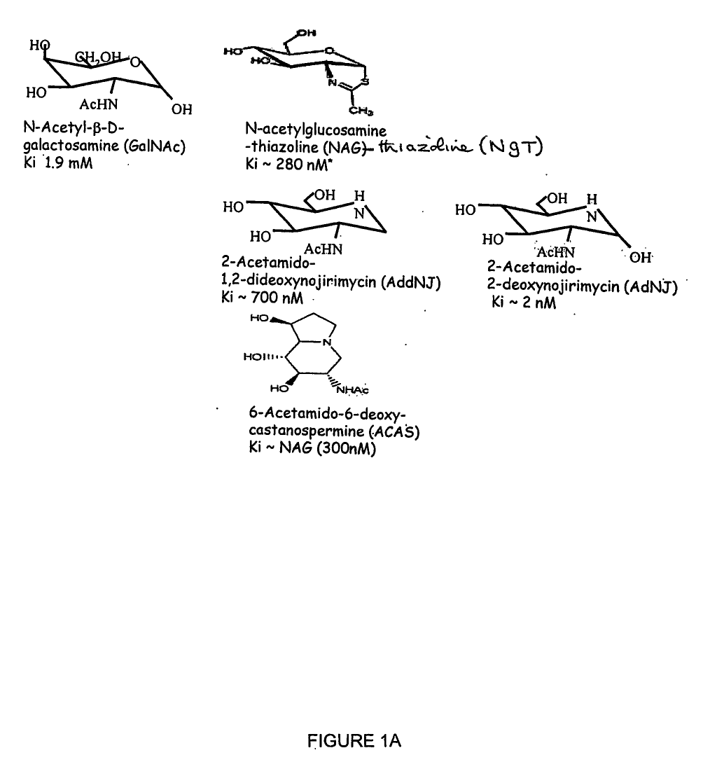

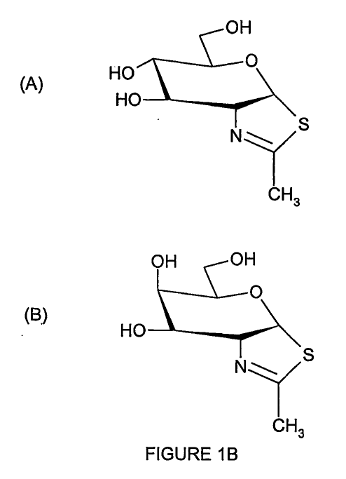

Suitable compounds for use in the methods and compositions of the invention include those compounds shown in FIGS. 1A, 1B and Tables 1 and 2.

Cells from patients with adult Sandhoff disease also showed enhanced activity of hexosaminidase A when treated with inhibitors of hexosaminidase. Possibly, newly synthesised mutant β subunit monomer is stabilised by the inhibitor and combines in an increased amount with the a subunit to give active hexosaminidase A.

The methods and pharmaceutical compositions of the invention are applicable to diseases resulting from any mutation in the α or β subunit of hexosaminidase A or B which produces an intact protein with residual activity, as is found in adult Tay-Sachs disease and in many cases of juvenile Tay-Sachs disease, and in adult Sandhoff disease, that can be stabilised by a compound which binds specifically to the hexosaminidase either inside or outside the enzyme active site.

The results described herein indicate that inhibitor treatment increases the amount of mutant hexosaminidase A activity and protein in the lysosomes of treated ASTD cells.

In the acute infantile form of Tay-Sachs disease and in infantile Sandhoff disease, where the hexosaminidase A protein is absent, this treatment is not applicable.

The total activity of hexosaminidases A, B and, where present, for example in Sandhoff disease), S may be determined, for example, by their ability to hydrolyse the fluorogenic substrate 4-methyl-umbelliferyl-N-acetyl βD-glycosaminide (MUG) and hexosaminidase A and S activity may be specifically measured using 4-methylumbelliferyl-βD N-acetylglucosamine-6-sulphate (MUGS) as described in Bayleran et al., (1984), Clin. Chem. Acta, v. 143, p. 73. Competitive inhibitors of hexosaminidase A or B activity useful in the methods and compositions of the invention may be identified using the MUG/MUGS assays, as described in Knapp et al., (1996), J. Am. Chem. Soc., v. 118, p. 6804 and Panday et al., (2000), Helv. Chim. Acta, v. 83, p. 1205. Suitable inhibitors include N-acetyl glucosaminide and N-acetyl galactosaminide derivatives having a C-2 acetamido and a C-5 hydroxy methyl group and compounds which mimic the cyclised oxazolinium ion which is a reaction intermediate of the Family 20 enzymes which include lysosomal hexosaminidase, for example N-acetylglucosamine-thiazoline.

N-acetyglucosamine-thiazoline and N-acetyl galactosamine-thiazoline are examples of compounds effective in the methods and compositions of the invention. Acylated derivatives of these compounds may also be used, for example C1 to C20 acyl derivatives, for example C1 to C10 acyl derivatives. Derivatives may contain from 1 to 3 acyl groups. In one embodiment, acetyl derivatives of these compounds are employed.

Compounds which improve the stability of hexosaminidase although not competitive inhibitors of the enzyme may be identified using the ATSD cell culture system described in the examples.

Hexosaminidase enhancers may be administered to a subject in need of treatment either alone or along with a pharmaceutically acceptable carrier; administration may, for example, be oral or parenteral, intravenous or subcutaneous. The enhancers may be formulated in liposomes for administration. Suitable methods of formulation are known to those of skill in the art and are described in texts such as Remington's Pharmaceutical Sciences (Mack Publishing Company, Easton, Pa. U.S.A. 1985). A serum level of enhancer compound in the range from 0.01 μM to 100 μM should be aimed for, preferably in the range from 0.01 μM to 10 μM. Those of skill in the art are able to determine dosages suitable to achieve such serum levels of inhibitor. Where the enhancer compound is a hexosaminidase inhibitor, serum levels of inhibitor should be monitored to avoid reaching inhibitory levels which will reduce hexosaminidase activity once it enters the lysosome, or to signal that inhibitory levels have been reached, in which case administration of the inhibitory compound may be reduced. Serum inhibitor levels may be monitored, for example, using the method described by Conzelman et al., (1982), Eur. J. Biochem., v. 123, p. 455).

The data described herein indicate that the methods and pharmaceutical compositions of the invention are likely to provide a sufficient increase in hexosaminidase activity to give amelioration of hexosaminidase deficiency-related diseases.

In a further embodiment, the treatments described herein, using compounds which enhance hexosaminidase activity, may be used in combination with “substrate deprivation” therapy. This should permit the use of lower doses of NB-DNJ, with reduced toxicity.

Adult onset and juvenile Tay-Sachs disease patients are candidates for treatment by the methods and compositions of the invention. As known to those skilled in the art, juvenile and adult onset Tay-Sachs may be diagnosed through a combination of physical symptoms and determinations of hexosaminidase A activity.

Compounds which inhibit hexosaminidase A activity are likely also to inhibit hexosaminidase B activity. Such compounds are therefore likely to stabilise both the alpha and beta subunits of the enzyme. The methods and compositions of the invention may also therefore be used to treat adult onset and juvenile forms of Sandhoff disease, where there is a mutation which destabilises the hexosaminidase β subunit.

In view of the close similarity between the substrates recognised by hexosaminidases A and B and those recognised by other lysosomal glycohydrolases, many of the inhibitors described herein may inhibit other lysosomal glycohydrolases. The methods of the invention may therefore be used to treat diseases associated with reduced activity of an enzyme closely related to hexosaminidase, for example San Phillipo disease Type B (reduced α-N-acetyl glucosaminidase activity), and Morquio disease (β galactosidase).

The inventors have found also that the compounds described herein protect hexosaminidase A against heat denaturation and increase its heat stability, which will also lead to improved hexosaminidase A activity. Compounds may also be used which bind away from the active site but serve to stabilise the hexosaminidase enzymes against thermal denaturation, thus acting as chemical chaperones.

In a further embodiment, the invention provides a method for screening a candidate compound for its ability to stabilise a subunit of hexosaminidase or to increase hexosaminidase activity in a cell. A candidate compound may be screened in a heat denaturation assay as described in the examples herein, looking for increased heat stability of hexosaminidase in the presence of the candidate compound. Alternatively, a cell line such as the ATSD cell lines described herein may be treated with the candidate compound, by the methods described herein, and the hexosaminidase activity of the treated cells compared to that of control cells, an increased activity identifying an active compound.

The invention further includes compounds identified by the above-described screening methods as stabilisers of hexosaminidase or compounds which increase hexosaminidase activity.

### EXAMPLES

Materials and Methods

Fluorogenic substrates MUG and MUGS were purchased from SIGMA. Rabbit polyclonal antibodies (Ab) against Human hexosaminidase A were prepared as previously described (Brown, 1993 #1566). Donkey polyclonal Ab developed against a C-terminal Calnexin peptide was purchased from Santa Cruz Biotechnology (California, USA). Castanospermine, Deoxynojirimycin, 2-acetamido 6-deoxycastanospermine (IRL, New Zealand), 2-acetamido-1,2-dideoxynojirimycin (AddNJ) (TRC, Toronto, CANADA), and N-acetyl-β-D-galactosamine (GaINAc) (SIGMA) were commercially available; 2 acetamido-2-deoxynojirimycin (AdNJ) and NAG thiazoline (NGT) were synthesised and purified according to Kappes and Legler, (1989) and Knapp et al., (1996) J. Amer. Chem. Soc., v. 118, pp. 6804-6805 respectively. NAGal-thiazoline was synthesised by a method analogous to that for NGT, using a comparable galactose derivative as starting material. All compounds were dissolved in water and used as 10-25 mg/ml solutions.

Cell Lines

Fibroblast cell lines from an unaffected female patient (WT, 4212), from a 40 year old female patient diagnosed with the chronic (adult) form of TSD and homozygous for the mutation G269S (ATSD, 1766 or 17662) and from a female fetus with the acute (infantile) form of TSD (ITSD, 2317) were grown in a-MEM (Invitrogen) supplemented with 10% FCS, and antibiotics Pen/Strep (Invitrogen) at 37° C. in a CO₂ incubator. 17662: A fibroblast cell line (17662) from an adult-onset Tay-Sachs patient homozygous for the most common point mutation associated with the disease, Gly 269 Ser in the α subunit of hexosaminidase A, was obtained from Department of Pediatrics, University of Saskatoon.

Wild type fibroblasts and other Tay-Sachs and Sandhoff cell lines were obtained from Hospital for Sick Children cell culture facilities, Toronto.

Cell Culture and Hexosaminidase Assay

The effect of inhibitors on hexosaminidase A activity in fibroblast cells was evaluated using two formats (96- or 6-wells). For the dose response curves and kinetics of increased hexosaminidase A activity in the presence or absence of each compound, cells grown in 96 well tissue culture plates (Falcon) were used. To ensure that equal numbers of cells were seeded in each well, trypsinized cells were diluted to give 50% confluence when plated and 200 μL aliquoted into each well of the plate. Cells grown for longer than 5 days were supplemented with fresh medium. After allowing one day for the cells to attach, inhibitory compounds to be evaluated for activity were diluted in medium and filter sterilised (Millipore). Each concentration point of the compounds was evaluated in triplicate.

Following 3-7 days of incubation at 37° C. in the presence or absence of the compound being tested, intracellular hexosaminidase A activity was determined. Medium was removed, cells were washed with PBS twice and lysed using 60 μL of 10 mM citrate phosphate buffer pH 4.5 (CP buffer) containing 0.5% human serum albumin and 0.5% Triton X-100. Cells were solubilized at room temperature for 15 min, and subsequently 25 μL of lysate was transferred to a new 96 well plate. Hexosaminidase A activity in lysates was measured using 25 μL of 3.2 mM MUGS in CP buffer with incubation at 37° for 1 hr. Afterwards, 250 μL of 0.1M 2-amino-2-methyl-1-propanol (pH 10.5) was used to stop the reaction and increase the fluorescence of the methyl umbelliferyl product, Fluorescence was read with a Perkin-Elmer LS50B Luminescence Spectrometer equipped with a sipper and using excitation wavelength of 365 nm and emission wavelength of 450 nm. For dose response and kinetic experiments, the relative increase in hexosaminidase A activity was expressed as the average fluorescence reading from three or four wells, with cells grown in the presence of compound divided by the average fluorescent reading from a minimum of four wells, with cells grown in the absence any compound. To control for plate to plate variability, control (untreated) cells were included with each 96 well plate.

For western blot analysis, Cellulose acetate eletrophoresis, and to determine the increase in hexosaminidase A specific activity, ATSD fibroblasts were grown for 5 days in 6 well tissue culture plates (Falcon, 40 mm2) containing 1.5 mL a-MEM, FCS P/S media supplemented with/without the compounds to be evaluated. Subsequently, media were removed, cells were washed twice in PBS, and finally scraped into 1 mL of PBS. Following pelleting in microfuge, the cells were resuspended in 10 mM phosphate buffer pH 6.1 containing 5% glycerol and disrupted using by sonication (ARTEK Sonic Dimembrator, Farmington N.Y.), on ice with 4 pulses for 10 seconds at setting 6. Cleared lysates were prepared by microcentrifugation (Eppendorf) at maximum setting for 15 min at 4° C. and total protein concentration was determined using BCA protein assay (PIERCE) according to manufacturers instructions. Hexosaminidase A activity was determined using MUGS substrate at 37° C. for 1 hr as described above and expressed as nmoles of MU released/hr/mg of total protein.

Western Blotting

Lysates containing 5 μg total protein were subjected to PAGE on a 10% bis:acrylamide gel, electrophoretically transferred to nitrocellulose (Schlicher and Schull), blotted with 5% non-fat dry skim milk powder in 25 mM Tris pH 7.5, 150 mM NaCl, 0.025% Tween 20 buffer overnight at room temperature. Blocked blots were incubated with rabbit anti-human hexosaminidase A polyclonal Ab, washed with blocking buffer, followed by incubation with anti-rabbit IgG peroxidase conjugated secondary Ab. Blots were developed using chemiluminescent substrate according to manufacturers protocol (Amersham) and recorded on BIOMAX X-ray film (KODAK).

Cellulose Acetate Electrophoresis (CAE)

To directly visualise hexosaminidase A heterodimers, CAE was performed as follows. Briefly, lysates containing 2 μg of total protein were spotted on Sepraphore (Gelman) cellulose acetate strips (prewetted in 20 mM sodium phosphate buffer ph 7.0) and partially dried. Samples were resolved electrophoretically at 10 mA for 20 min. Electrophoresed strips were overlaid with another cellulose acetate strip soaked in 3.2 mM MUG, wrapped in plastic wrap, and incubated for 1 hr at 37° C. Subsequently, strips were briefly incubated over an ammonium hydroxide solution. Bands corresponding to released methyl umbelliferyl were visualised and photographed under UV light (340 nm).

Heat Inactivation Assay

For heat inactivation experiments, purified placental hexosaminidase A or partially purified hexosaminidase A from unaffected or ATSD fibroblasts were used. Partially purified hexosaminidase A was prepared from sonicated lysates from unaffected and ATSD fibroblasts in 10 mM sodium Phosphate buffer pH 6.1 5% glycerol. Lysates were applied to DEAE Sepharose columns previously washed with 1M NaCl 10 mM Na phosphate, pH 6.1 and equilibrated with 10 mM Na phosphate buffer pH 6.1. The column was washed with 10 column volumes of 20 mM NaCl, Na Phosphate buffer pH 6.1. This fraction which was not collected contained the hexosaminidase B isozyme. Hexosaminidase A was eluted and fractions collected using 100 mM NaCl Na Phosphare buffer pH 6.1. For heat inactivation experiments, equal amounts of total protein from WT and mutant hexosaminidase A fractions were diluted three-fold in 10 mM citrate phosphate buffer pH 4.5 containing 0.5% Human serum albumin. Stability of the WT and mutant hexosaminidase A enzymes in the presence or absence of hexosaminidase A inhibitors were evaluated at 42° C. in Eppendorf tubes containing 25 or 50 μL of diluted enzyme. Following incubation, tubes were placed on ice until all time points were collected. Subsequently, the heat treated samples were equilibrated to 37° C. for 10 min, followed by addition of MUGS substrate and incubation at 37° C. for further 30-60 min. Fluorescent readings were obtained as described above.

Subcellular Fractionation

A lysosomal fraction was prepared from NAG-Thiazoline (NGT) treated and untreated ATSD fibroblasts using a modification of a protocol described in Marsh et al. (1987). Following a 7 day incubation in growth medium containing or lacking 250 μg/mL of NAG thiazoline, cells from twenty 150 mm tissue culture plates were washed and scraped into PBS and pelleted at 100 g. The pellet was resuspended in basic medium (10 mM triethanolamine, 10 mM Acetic Acid, 1 mM EDTA and 0.25M sucrose pH 7.4) and cells homogenised with 10 strokes of a tight fitting Dounce homogenizer. The homogenate was centrifuged for 10 min at 1000 g, the supernatant put aside and the pellet was re-homogenised, spun and the resulting supematant was pooled with the first. The pooled supernatants were again centrifuged at 1000 g. The resulting supematant was overlaid onto a 1M sucrose (in 10 mM Triethynolamine, 10 mM Acetic acid pH 7.4) cushion and centrifuged in a SW41Ti rotor at 100,000 g for 35 min. The pellet was resuspended and diluted four fold with 10 mM Triethanolamine 10 mM acetic acid. A Bradford protein assay was performed on the suspension to determine total protein. To limit aggregation of lysosomes, TPCK Trypsin was added at 2% (wt/wt) to the suspension followed by incubation at 37° C. for 1 hr, and sequential filtration through 5μ and 3μ filters and finally centrifugation for 10 min. at 1000 g. The resulting supernatant was used as a lysosomal fraction.

### Example 1

17662 cells and wild type fibroblasts were cultured as described above in the presence of 10-100 μg/ml of one of the following:

(i) N-acetyl-β-D-galactosamine (GalNAc);

(ii) 6-acetamido-6-deoxy-castanospermine (ACAS);

(iii) N-acetylglucosamine-thiazoline (NAG-thiazoline or NGT);

(iv) 2-acetamido-1,2-dideoxynojirimycin (AddNJ);

(v) 2-acetamido-2 deoxynojirimycin (AdNJ);

(vi) deoxynojirimycin (DNJ); or (vii) castanospermine (CAS).

The structures of these compounds are shown in FIG. 1. With the exception of (vi) and (vii), all contain an acetamido group which acts as a non-enzymic nucleophile in the hydrolysis of the substrate. NAG-thiazoline is a stable thiazolium which mimics the internal oxazolium ring normally formed as the reaction intermediate. Compounds (i) to (v) are hexosaminidase inhibitors. DNJ and CAS, which are inhibitors of α-glucosidase I and II which produce the glycan substrates recognised by-the ER resident chaperone calnexin, served as negative controls.

The results with 17662 cells are shown in FIG. 2. Culture in the presence of a hexosaminidase inhibitor resulted in a 3 to 6 fold increase in hexosaminidase A activity relative to untreated 17662 cells. When wild type fibroblasts were similarly treated, a 20-50% increase hexosaminidase A activity relative to untreated cells was seen (data not shown).

### Example 2

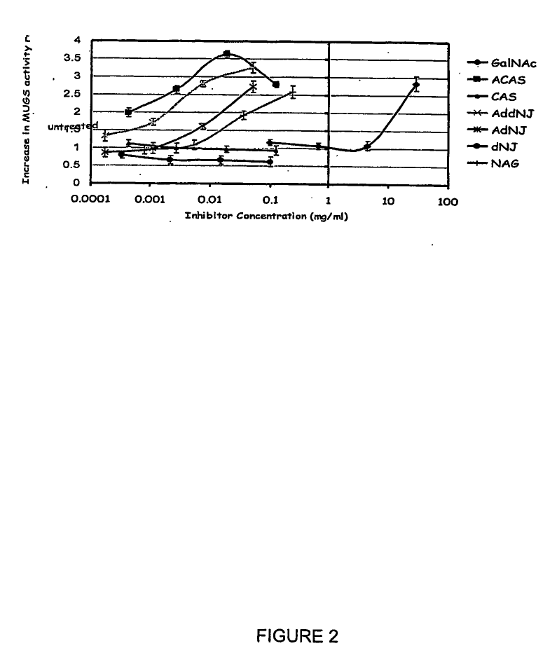

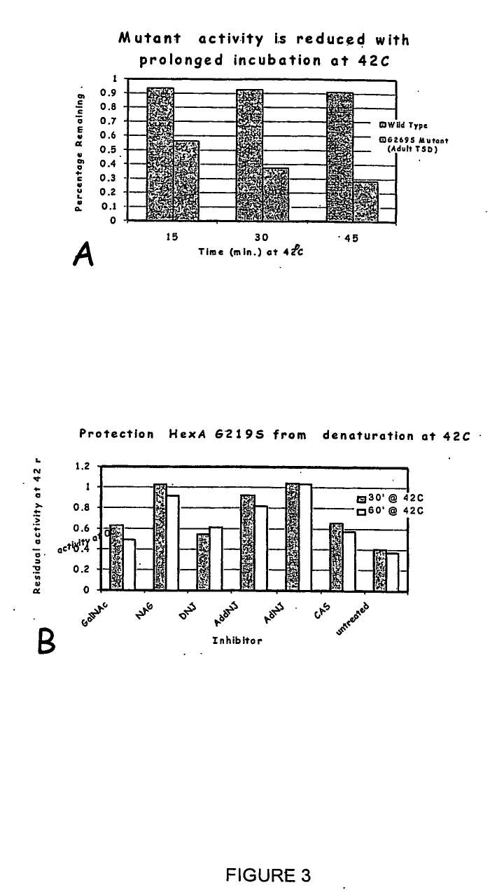

Hexosaminidase A was partially purified by DEAE ion exchange chromatography from a hypotonic lysate of 17662 or Wild Type fibroblasts cells. Heat inactivation kinetics of mutant and WT enzyme were performed (O'Brien et al., (1970), N. Eng. J. Med., v. 283, p. 15) using eluate containing hexosaminidase A activity (MUG/MUGS ratio 5:1) which was diluted with 0.1M citrate buffer pH 4.5 and incubated at 0° C. at 42° C. for 15, 30 or 45 minutes, with subsequent return to 0° C. Remaining hexosaminidase A activity was monitored using MUGS. The results are shown in FIG. 3A.

Whereas >90% wild type enzyme activity remained after 45 min. at 42° C., <50% of mutant hexosaminidase A activity remained after 30 min, i.e. its half life was reduced to about 30 min, in contrast to the wild type half life of 300 min.

For inhibitor experiments, inhibitors were diluted to a concentration which reduced enzyme activity by 50%. Inhibitors were then added to the mutant hexosaminidase A eluate and incubated at 0° C. or 42° C., followed by a MUGS activity assay. The results are shown in FIG. 3B. In the presence of several inhibitors (NAG, AddNJ, AdNJ), the half life of the mutant hexosaminidase was restored to near wild type levels.

### Example 3

17662 cells were grown in 6 well tissue culture dishes in medium with or without inhibitor for 6 days. Subsequently, medium was removed, cells were washed twice with PBS, scraped off into 1 ml PBS, centrifuged, and pellet was resuspended in 10 mM potassium phosphate buffer pH 6.1, 1% Triton X100. Half of the aliquot was used in a western blotting experiment and the other half was used to determine the MUGS activity of the sample. For western blotting, following PAGE, transfer to nitrocellulose and blocking in non-fat dry milk, the blot was incubated sequentially with polyclonal anti-rabbit hexosaminidase A antibody and goat anti-rabbit IgG peroxidase-conjugated secondary antibody (Amersham Biosciences). Antibody binding was visualized by ECL according to the manufacturer's instructions for Amersham ECL.

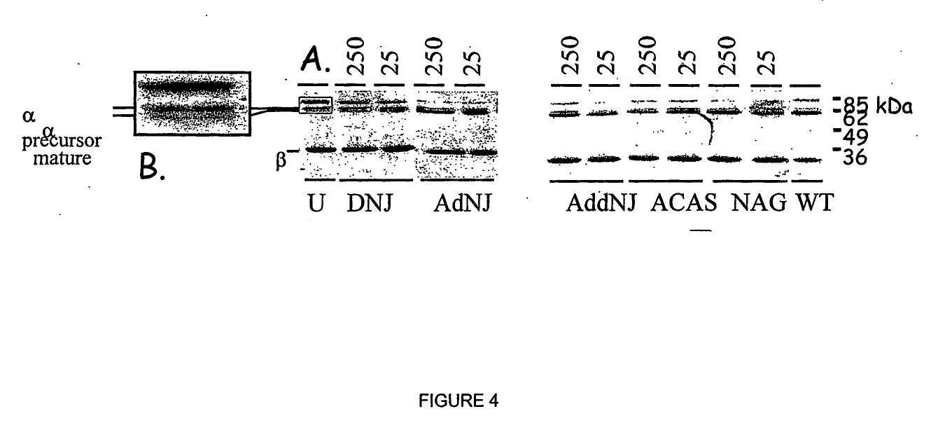

The results are shown in FIG. 4. The increase in hexosaminidase activity with inhibitor treatment was accompanied by an increase in a subunit protein in the cells (FIG. 4A). As seen in FIG. 4B, the increased levels of a protein of 60 kd correspond to the α-subunit of the enzyme, and are consistent with the size expected for an α-subunit which has been transported to the lysosome and processed to the mature form.

### Example 4

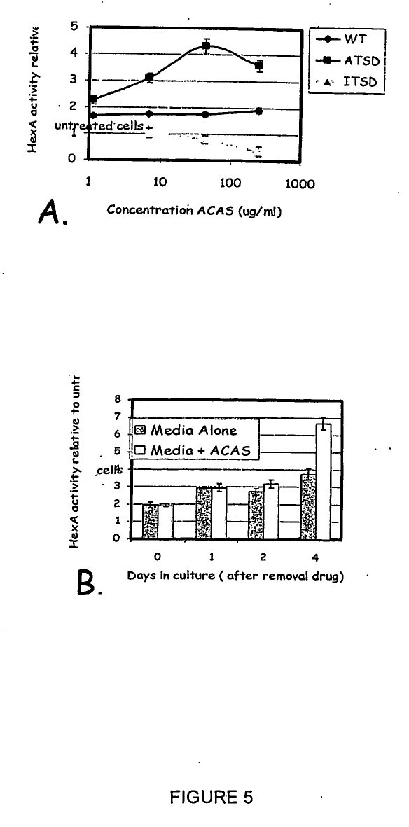

To determine the specificity of inhibitors, fibroblasts derived from an asymptomatic patient (WT), an adult onset Tay-Sachs patient (17662) and an infantile Tay-Sachs patient with a hexosaminidase A null mutation, were grown in 96 well plates and incubated with medium containing ACAS for 4 days. Subsequently, hexosaminidase A activity was assayed using a MUGS assay as described above. The results are shown in FIG. 5A. To determine whether the increased effect of the inhibitor persists, after incubation of replicate rows of 17662 cells in the presence of ACAS (25 μg/ml). for 3 days, medium containing inhibitor was removed from half of the rows and replaced with inhibitor-free medium. After an additional 0, 1, 2 or 4 days culture, a hexosaminidase A assay was performed on the cells. The results are shown in FIG. 5B. In all cases, each data point represents the average activity from three adjacent wells.

As seen from FIG. 5A, the inhibitor increased hexosaminidase A activity in 17662 cells and wild type cells but not in the cells from the infantile Tay-Sachs patient; where only hexosaminidase B is present. As seen from FIG. 5B, the restorative effect of ACAS on hexosaminidase A activity persisted in 17662 cells for at least 4 days after removal of the inhibitor and growth in inhibitor-free medium.

### Example 5

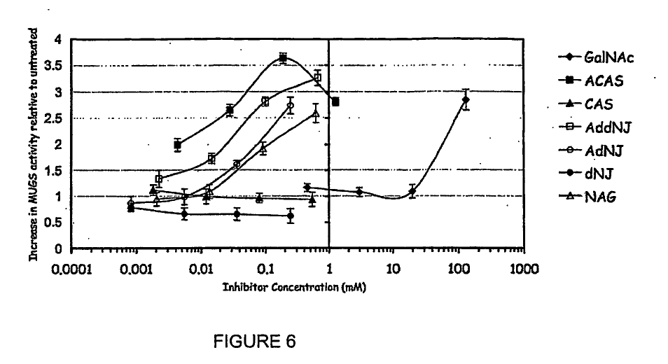

Dose Response of Chronic TSD Fibroblasts to Inhibitors (FIG. 6).

In fibroblasts from a chronic TSD patient homozygous for the aGly269Ser, hexosaminidase A activity found to be ˜10% of normal (data not shown). After five days of growth in the presence of the compounds listed in FIG. 6, increased hydrolysis of MUGS was observed in lysates from cells treated with GalNAc, AddNJ, ACAS and NGT. Cell lysis occurred when concentrations of GalNAc were >200 mM. A decrease in hexosaminidase A activity was found when ACAS was used at concentrations of >200 μM which was associated with a decrease in the number of cells. The decline in effectiveness with decreasing concentration of inhibitors was greatest for GalNAc and least for ACAS, which was still effective in enhancing hexosaminidase A activity even at concentrations of 5 μM.

Enhanced Hexosaminidase A Activity Following Removal of ACAS and NGT from the Growth Media.

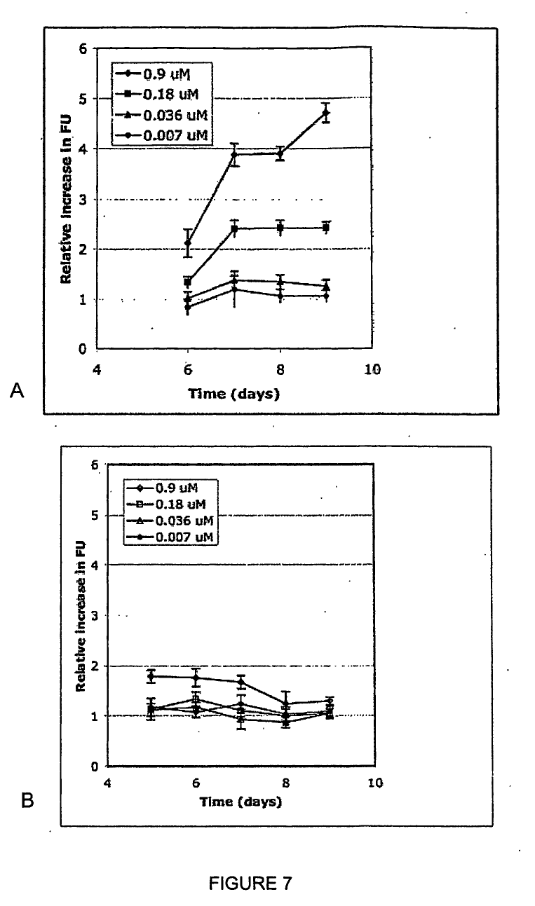

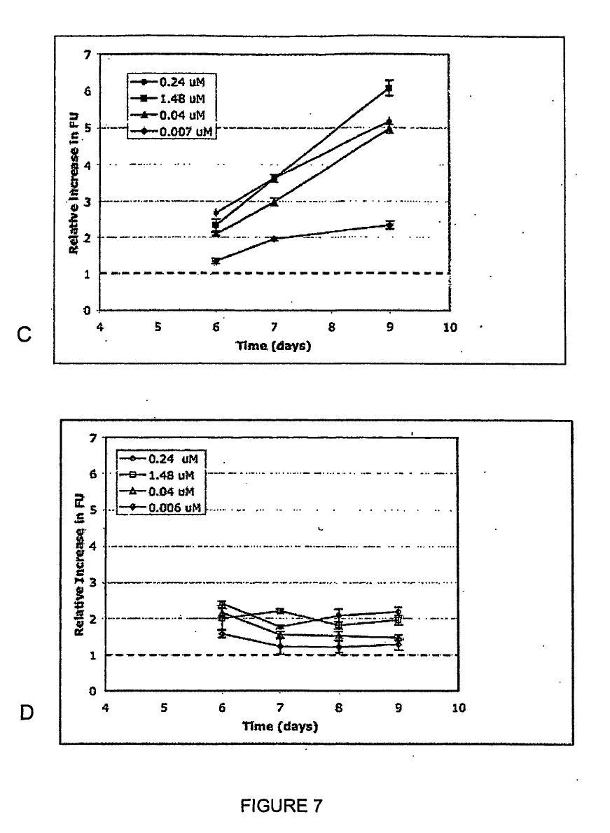

The kinetics of hexosaminidase A enhancement by ACAS and NGT were more closely examined. FIGS. 7A and 7C follow hexosaminidase A activity of ASTD fibroblasts with increasing duration of incubation in the presence of NGT and ACAS respectively. It is interesting to note that hexosaminidase A activity continued to increase with increasing incubation times at all concentrations of ACAS. However, only at the highest concentrations of NGT ( 0.9 mM) did hexosaminidase A activity continue to increase. At lower concentrations of NGT (0.18 mM, 0.03 mM and 0.007 mM), the enhancing effects on hexosaminidase A activity peaked after seven days incubation, and plateaud or declined thereafter.

We next determined if the observed increased hexosaminidase A activity persisted for a period after the compounds had been removed from the culture medium. ATSD fibroblasts which had been grown in the presence of NGT or ACAS for 4 days continued to show enhanced hexosaminidase A activity even after 1-4 days of growth in medium lacking the compounds (FIGS. 7B and 7D respectively). The enhancing effect of ACAS persisted for longer than 3 days, whereas the effect of NGT was reduced to near background levels after 2 days of growth in normal medium.

Specificity of Enhanced Hexosaminidase A Activity

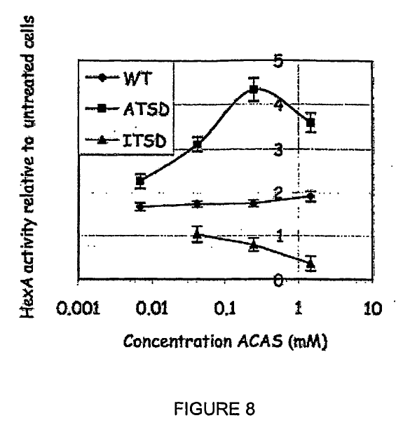

In order to demonstrate that the increased MUGS hydrolysis was due to an increase in the hexosaminidase A isozyme, the effect of NGT and ACAS on hexosaminidase A activity in unaffected fibroblasts (WT) and fibroblasts from a fetus with the acute (infantile) form of Tay Sachs (ITSD) were evaluated (FIG. 8). The ITSD cells do not produce any a-protein (data not shown). Unlike ATSD fibroblasts treated with NGT, a decline in MUGS hydrolysis (likely from inhibiting residual hexosaminidase B activity towards this substrate) was seen with increasing concentration of the compounds in ITSD cells. In the case of WT fibroblasts, a significant two fold increase in hexosaminidase A activity was seen only when concentrations of NGT reached 3 mM. In contrast, treatment of either WT orISTD fibroblasts with ACAS resulted in decreased hexosaminidase A activity at all concentrations. These results indicate that the increased hexosaminidase A activity in ATSD fibroblasts treated with the compounds is due to increased hexosaminidase A activity of the mutant protein.

Treatment of ATSD Fibroblasts Results in Increased Levels of the Lysosomally Processed (Mature) α-subunit and the Hexosaminidase A Heterodimer.

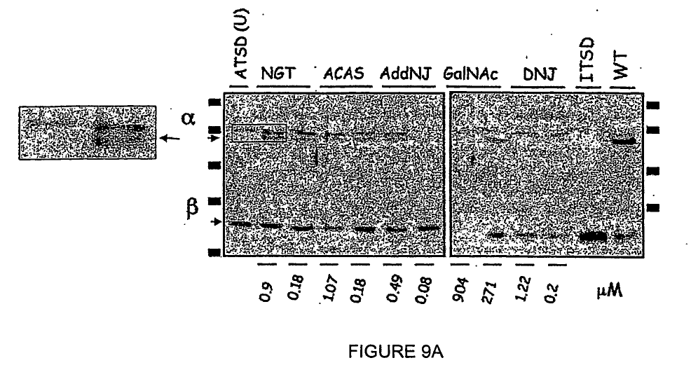

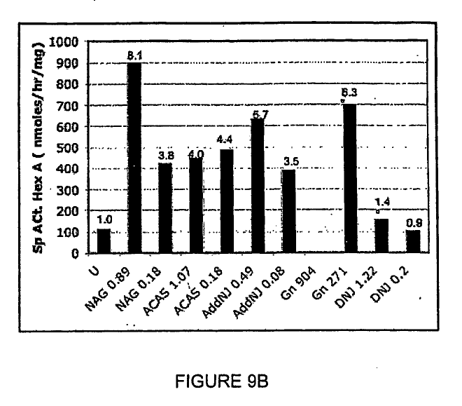

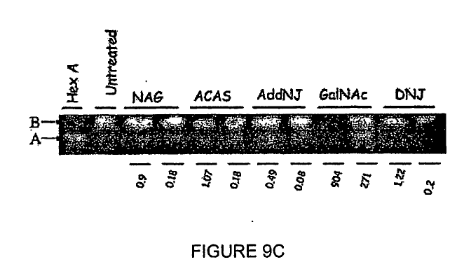

To show directly that treatment of ATSD fibroblasts with the inhibitory compounds resulted in increased amounts of the α-subunit in the lysosome, cell lysates were subjected to Western blotting with an anti-hexosaminidase A antibody (FIG. 9A). The results show that in comparison to untreated cells, increased amounts of a band migrating at 56 kD corresponding to lysosomally processed α-subunit were seen in cells treated with AddNJ, GalNAc, NGT and ACAS. The corresponding band is seen in WT fibroblasts but not in ITSD fibroblast cells. With the exception of cells treated with DNJ, the bands at 25 kD, corresponding to the lysosomally processed β-subunit of hexosaminidase, remain unaffected by the treatments.

The histogram of FIG. 9B demonstrates that these data closely correspond to the observed increases in specific activity of hexosaminidase A in ATSD-cells treated with the same compounds. ATSD cells treated with 0.9 mM NGT show the greatest increase in specific hexosaminidase A activity (eight fold) and levels of mature α-subunit.

To rule out the possibility that the observed increased MUGS hydrolysis in treated cells was due to hexosaminidase S, the different hexosaminidase isozymes in the lysates were resolved using cellulose acetate electrophoresis combined with MUG zymography. The results of FIG. 9C clearly show that there are increased amounts of a band A which co-migrates with one found in purified hexosaminidase A, but not detectable in lysates from untreated ATSD cells.

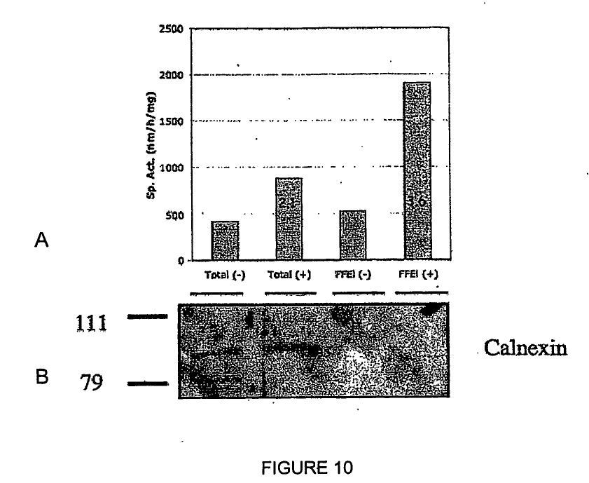

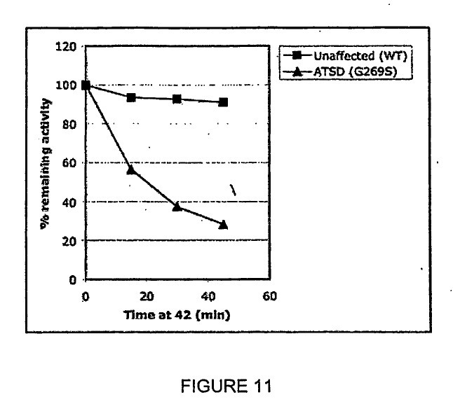

To demonstrate more directly that the increased hexosaminidase A activity is found in lysosomes, an enriched lysosomal fraction was prepared from NGT-treated and untreated ATSD fibroblasts. The results in FIG. 10A show that NGT treatment results in an approximately two fold increase in hexosaminidase A and that the specific activity of hexosaminidase A is further increased to 3.6 fold upon enrichment of the lysosomal fraction. As further confirmation that the fraction is enriched for lysosomes and does not contain any ER components, the lower panel, FIG. 10B, shows Western blots of the lysates prior to and following enrichment, probed with an antibody against Calnexin, a resident ER protein. The lysosomal enriched fraction does not contain detectable amounts of calnexin. The combined results in FIGS. 9 and 10 demonstrate that the increased hexosaminidase A activity observed in NGT-treated ATSD fibroblasts is from lysosome hexosaminidase A.

Compounds Binding to Hexosaminidase Protect Wild Type and Adult Mutant Hexosaminidase A from Thermal Denaturation.

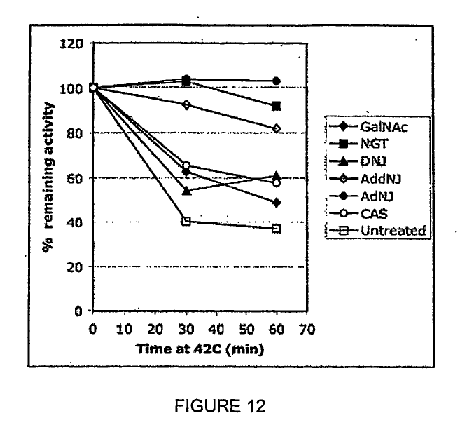

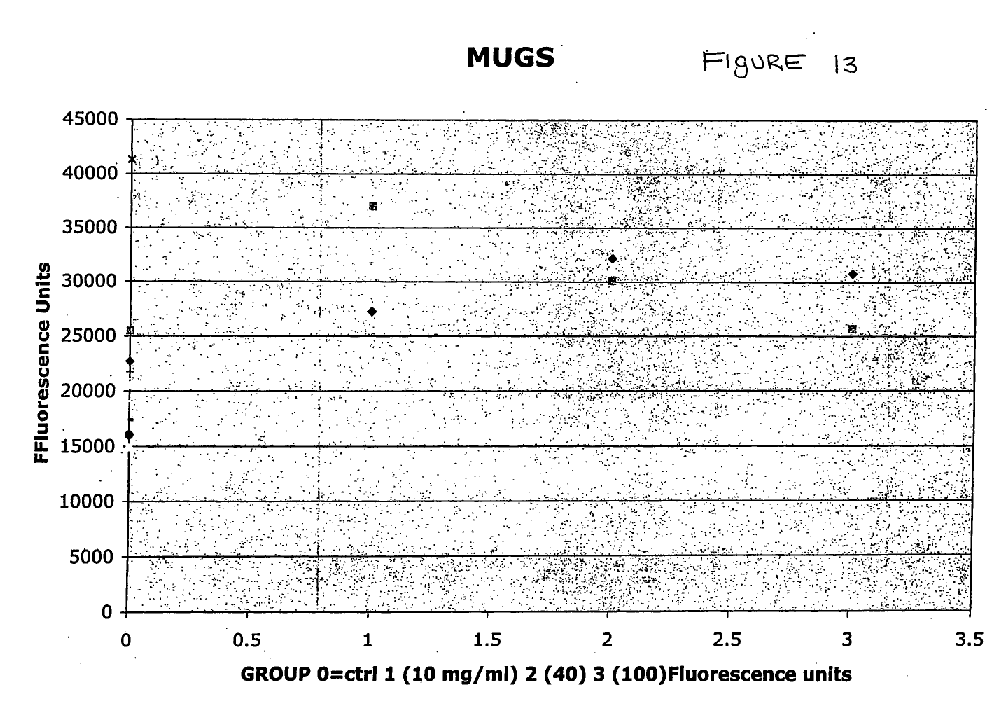

As shown in FIG. 11, both WT hexosaminidase and G269S mutant enzyme are susceptible to heat denaturation at 42° C. The results in FIG. 11 demonstrate that more than 50% of the activity of partially purified hexosaminidase A from ATSD fibroblasts is lost after 30 min., as compared to WT fibroblast hexosaminidase A which shows >20% reduced activity. WT and mutant hexosaminidase A were then incubated at 42° C. with the inhibitory compounds. For these experiments, the inhibitory compounds NGT and. AddNJ were added at concentrations resulting in 50% reduced hexosaminidase A activity. When mutant hexosaminidase A was incubated at 42° C. in the presence of compounds NGT and AddNJ, only a modest 10-20% reduction of activity was seen even after 60 minutes of incubation at 42° C. (FIG. 12). A similar protective effect of the compounds was seen when WT purified placental hexosaminidase A was incubated in the presence of ACAS, NGT or GalNAc (data not. shown).

### Example 6

Toxicity studies were carried out on adult mice by treating mice with 10 mg, 40 mg or 100 mg NGT by intraperitoneal injection; each treatment group contained 3 mice and untreated control group contained 10 mice.

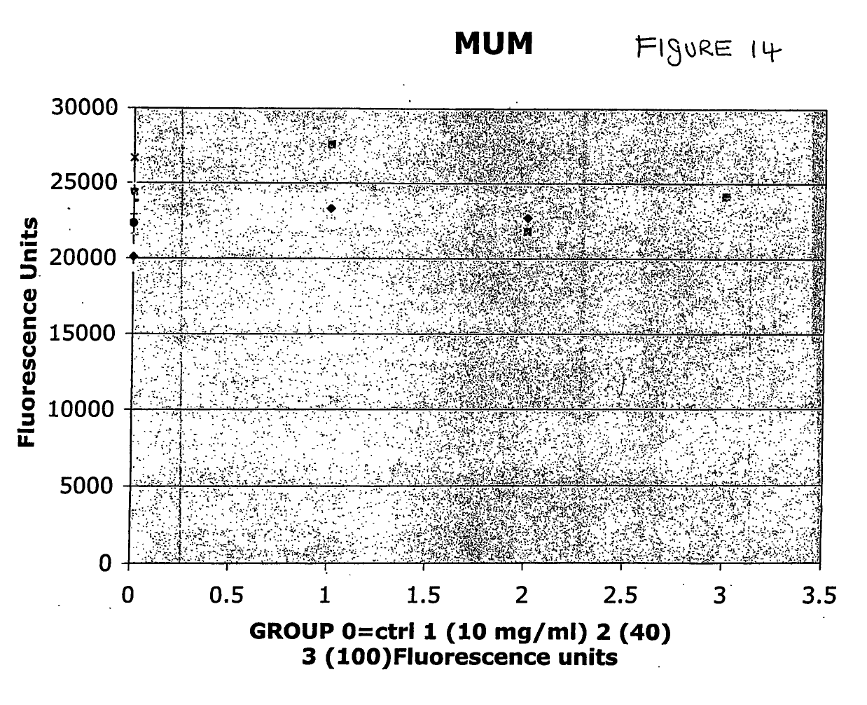

Serum levels of hexosaminidase A and βD mannosidase activities were measured as described above in each of the mice, 3 to 4 days after NGT treatment. As seen in FIG. 13, serum hexosaminidase A levels in treated mice were generally higher than those seen in the control group. As seen in FIG. 14, serum βD mannosidase levels were generally the same in control and treated mice. None of the mice treated with inhibitors showed any signs of toxicity.

Further toxicity studies were carried out in adult male CD1 mice using intravenous administration of NGT (40 mg/mouse) or sub-cutaneous administration, (40 mg/mouse every four days for up to 30 days)—data not shown. No behavioural differences were seen between treated and control groups and histological examination of autopsied tissues showed no changes in the treated mice. No acute or sub-acute toxicity was observed.

### Example 7

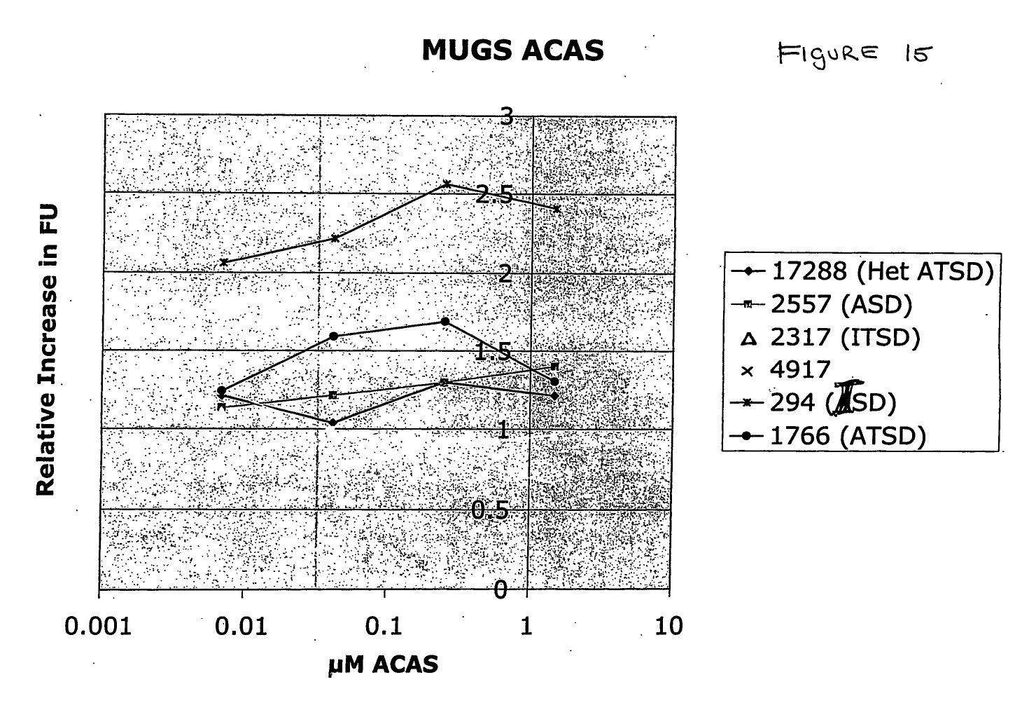

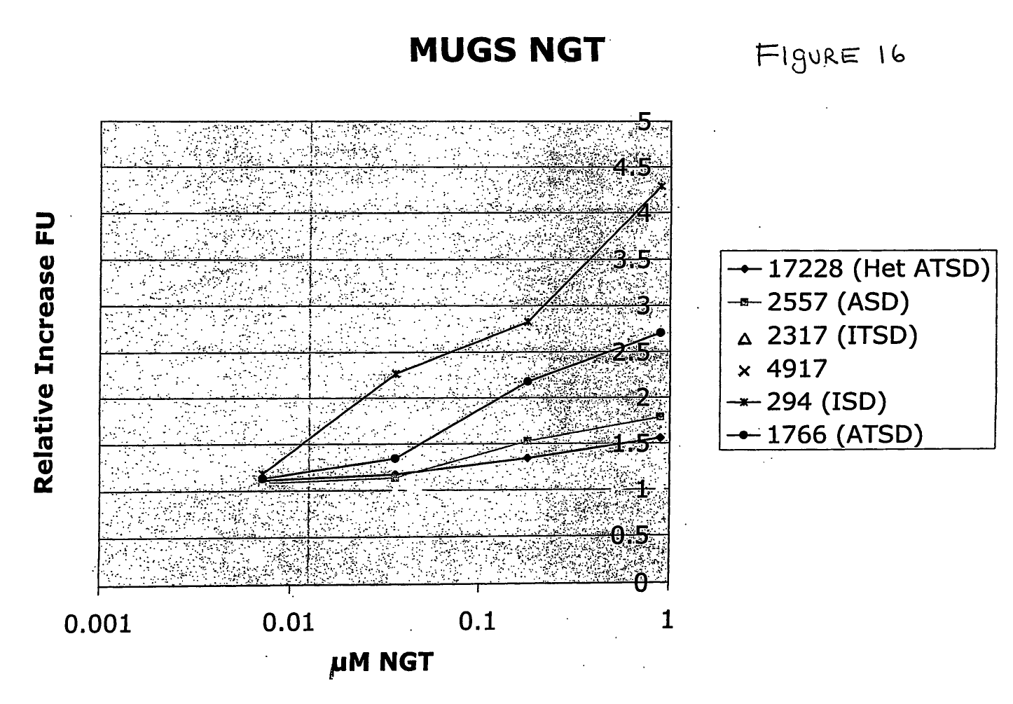

Fibroblast cell lines obtained from homozygous adult onset Tay-Sachs (ATSD), heterozygous adult onset Tay-Sachs (Het ATSD), infantile Tay-Sachs (ITSD and 4917), adult Sandhoff (ASD) and infantile Sandhoff (ISD) were cultured in the presence of various concentrations of ACAS and then examined for hexosaminidase A activity as described above. The results are shown in FIG. 15. ACAS treatment gave increased hexosaminidase A activity in ATSD, ASD and ISD cells. The same cell lines were cultured in the presence of NGT and their hexosaminidase A activity measured. The results are shown in FIG. 16. Again, increased hexosaminidase A activity was seen in ATSD, increased hexosaminidase A and S activity in ASD and increased hexosaminidase S activity in ISD cells.

### Example 8

Groups of 10 adult male CD1 mice were treated with an intraperitoneal injection of 10 mg, 40 mg or 100 mg NGT and 2 days later the treated mice and a control group of 20 saline treated mice were bled by intra-cardiac puncture. Plasma levels of hexosaminidases A plus B and hexosaminidase A alone were measured by the fluorescent assay described above, using MUG and MUGS as substrates respectively. β-mannosidase was similarly assayed using 4-methylumbelliferyl-β-D-mannopyranoside (MUM) as substrate.

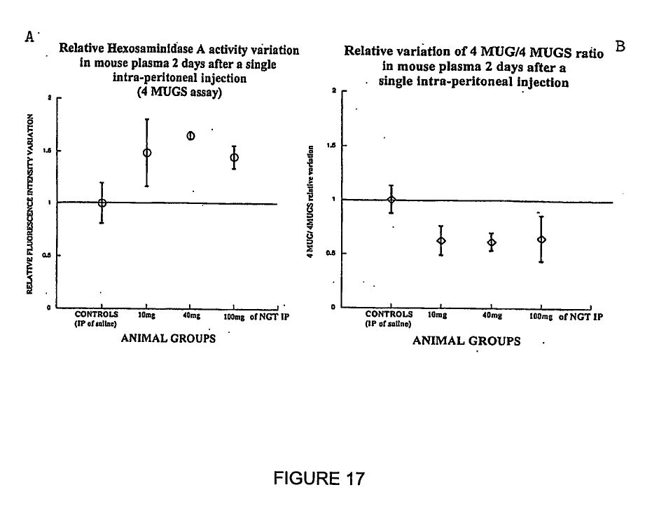

The results are shown in FIGS. 17A and B. Hexosaminidase A plus B (total Hex) and β-mannosidase levels were unchanged by NGT treatment. In contrast, hexosaminidase A increased and the ratio of hexosaminidase A plus B to hexosaminidase A decreased.

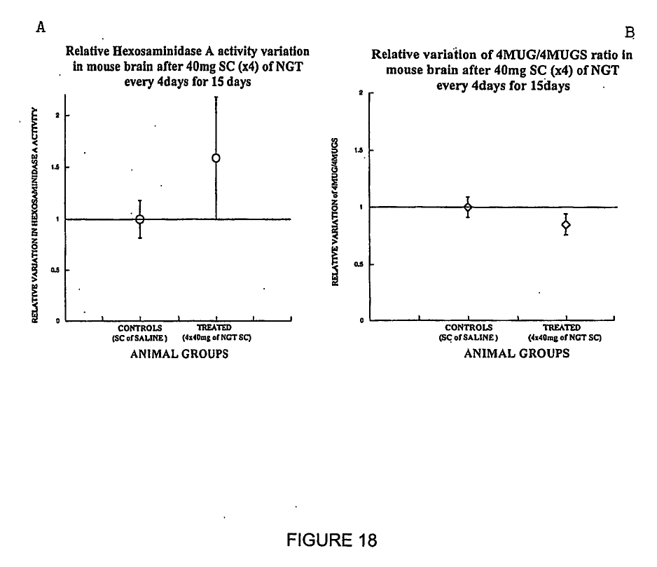

A further group of mice were treated with 40 mg NGT sub-cutaneously every 4 days for 15 days, brain tissue was collected after euthanasia and hexosaminidase A plus B and hexosaminidase A alone were measured. Again, hexosaminidase A activity increased while hexosaminidase A plus B was essentially unchanged. The results are shown in FIGS. 18A and B. These studies indicate that NGT does cross the blood-brain barrier.

### Example 9

Protection of Hexosaminidase B from Thermal Denaturation

Affinity purified, human placental hexosaminidase B was incubated at pH 4.5 with MUG as substrate in an assay similar to that described above, the mixture was adjusted to pH 10.0 and fluorescence was read essentially as described above. The effect on enzyme activity of various concentrations of NAG-thiazoline, NAGal-thiazoline and XylNAc—isofagomine.HCl was examined. Table A shows the Ki value of these inhibitory compounds on human hexosaminidases A and B and on hexosaminidase from the bacterial species Streptomyces plicatus (Sp. Hex.) Both NAG-thiazoline and NAGal-thiazoline are competitive inhibitors of both hexosaminidase A and B.

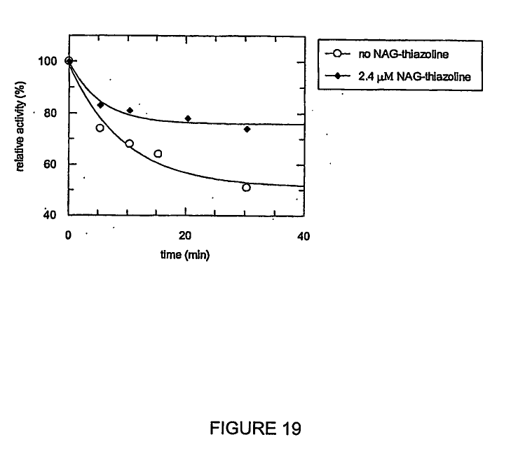

The effect of NAG-thiazoline on heat denaturation of human hexosaminidase B was also examined. The enzyme was incubated at 60° C. for up to 40 minutes in the presence or absence of 2.4 μm NAG-thiazoline and its activity was then assayed as described above using MUG as substrate. As seen in FIG. 19, the presence of NAG-thiazoline preserved greater hexosaminidase B activity than seen in the control.

NAG-thiazoline was also shown to protect hexosaminidase B against denaturation by guanidine hydrochloride.

### Example 10

ATSD (A,B) or ISD (CD) cells were treated with varying concentrations of NGT or GalNAcT for 2 days (ISD) or 5 days (ATSD). Cells were washed and lysed in Na Phosphate buffer pH 6.1. The lysates were divided into three equal aliquots (25 μl). To each aliquot, 25 μl of either MUGS ( 3.2 mM) or MUP (3 mg/ml) in 20 mM citrate phosphate buffer pH 4.3 was added. Reactions were incubated at 37° C. for 30-60 min. and stopped with 200 μl of 0.1M MAP buffer. Fluorescence was read using Molecular Devices.

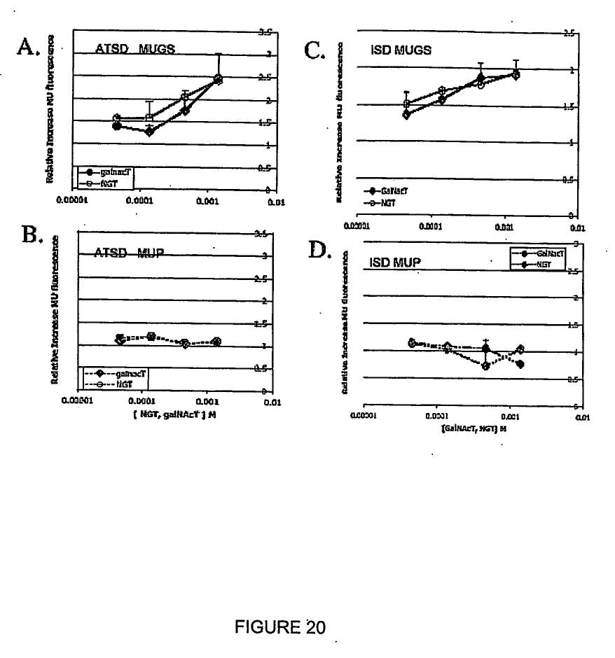

Gemini EM MAX with excitation and emission set to 365 nm and 450 nm, respectively. The activity of Hexosaminidase A/S was measured using MUGS hydrolysis and acid phosphatase activity was measured using methylumbelliferyl phosphate (MUP). The results are shown in FIG. 20. The two hexosaminidase inhibitors NGT and GalNAcT increased the activity of Hexosaminidase A/S in both ISD and ATSD cells but not the activity of the lysosomal enzyme acid phosphatase. Both appear to be equally effective in increasing hexosaminidase A/S activity.

### Example 11

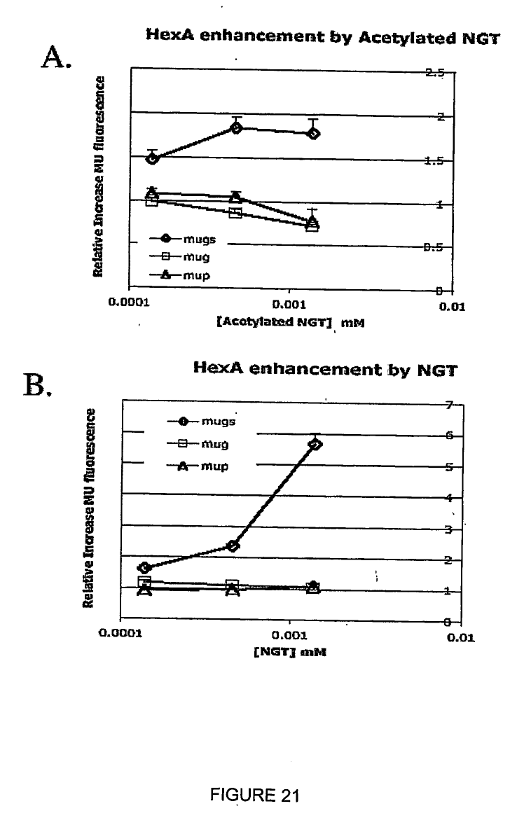

ATSD cells were treated with varying concentrations of NGT (B) or fully acetylated NGT (A) for 5 days. Cells were washed and lysed in Na Phosphate buffer pH 6.1. The lysates were divided into three equal aliquots (25 μl). The activity of total Hexosaminidase A/B/S Was measured using MUG hydrolysis whereas Hexosaminidase A/S was measured using MUGS hydrolysis; acid phosphatase activity was measured using methylumbelliferyl phosphate. To each aliquot, 25 μl of either MUGS (3.2 mM) or MUP ( 3 mg/ml) in 20 mM citrate phosphate buffer pH 4.3 was added. Reactions were incubated at 37° C. for 30-60 min. and stopped with 200 μl of 0.1M MAP buffer. Fluorescence was read using Molecular Devices Gemini EM MAX with excitation and emission set to 365nm and 450 nm, respectively. The results are shown in FIG. 21. The two hexosaminidase inhibitors increased the activity of hexosaminidase A in ATSD cells, but not the activity of the lysosomal enzyme acid phosphatase.

TABLE 1
Hexosaminidase Inhibitors

6-acetamido-6-deoxycastanospermine (Liu, Paul S., Kang, Mohinder S. and
Sunkara, Prasad S., (1991), Tetrahedron Letters, v. 52(6), pp. 719-720).
2-acetamido-2-deoxynojirimycin and 2-acetamido-1,2-dideoxynojirimycin
(Kappes, E. and Legler, G., (1989), J. Carbohydrate Chemistry, v. 8(3), pp.
371-388).
NAG-Thiazoline (Knapp, S., Vocadlo, D., Gao, Z., Kirk, B., Lou, J. and
Withers, S. G., (1996), J. Am. Chem. Soc., v. 118, pp. 6804-6805).
N-acetylglucosamine, N-acetylgalactosamine (Kapur, D. K. and Gupta, G. S.,
(May 15, 1986), Biochem. J., v. 236(1), pp. 103-109).
N-acetylglucosamine, Acetamide, N-acetylnojirimycin, N-2-Acetamido 2-
deoxyglucosylamine, N-acetylnojirimycin, N,N-dimethyldeoxynojirimycin, N-
acetylgluco-1,5-lactone, N-acetylglucolactam (Legler, G., Lullau, E., Kappes, E.
and Kastenholz, F., (Oct. 25, 1991), Biochem. Biophys. Acta, v.
1080(2), pp. 89-95).
2-acetamido 2-deoxy-D-gluconolactone (Conchie, J., Gelman, A. L. and Levvy, G. A.,
(June, 1967), Biochem. J., v. 103(3), pp. 609-615; Li, S. C. and Li, Y. T.,
(Oct. 10, 1970), J. Biol. Chem., v. 245(19), pp. 5153-5160).
NAGstatin (Aoyagi, T., Suda, H., Uotani, K., Kojima, F., Aoyama, T.,
Horiguchi, K., Hamada, M. and Takeuchi, T., (September, 1992), J. Antibot
(Tokyo), v. 45(9), pp. 1404-1408).
2-acetamido-1,4-imino-1,2,4-tridesoxy-D-galactitol (Liessem, B., Giannis, A.,
Sandhoff, K. and Nieger, M., (Dec. 16, 1993), Carbohydr. Res., v.
250(1), pp. 19-30).
N-acetylglucosaminono-1,5-lactone oxime and N-acetylglucosaminono-1,5-
lactone O-(phenylcarbamoyl)-oxime Horsch, M., Hoesch, L. Vasella, A. and
Rast, D. M., (May 8, 1991), Eur. J. Biochem., v. 197(3), pp. 815-818).
Iminocyclitol(Compound 4) (Liu, J., Shikhman, A. R., Lotz, M. K. and Wong, C. H.,
(July, 2001), Chem. Biol., v. 8(7), pp. 701-711).
N-acetylgalactosamine derived Tetrazole Heightman, T. D., Ermert, Ph., Klein, D.
and Vasella, A, (1995), Helv. Chim. Acta, v. 78, pp. 514-532).
Gualamycin (Tatsuta, K.; Kitagawa, M., Horiuchi, T., Tsuchiya, K. and
Shimada, N., (July, 1995), J. Antibot (Tokyo), v. 48(7), pp. 741-744).
Phenylsemicarbazones (Wolk, D. R., Vasella, A., Schweikart, F. and Peter, M. G.,
(1992), Helv. Chim. Acta, v. 75, p. 323).
N-acetylglucosamine related 1,2,3 and 1,2,4 triazoles (Panday, Narendra and
Vasella, Andrea, (2000), Helv. Chim. Acta, v. 83, pp. 1205-1208).
Nojirimycin based glycosidase inhibitors (c1999), Nojirimycin and Beyond,
publ. Weinheim, New York: Wiley-VCH).
N-acetyl glucosamine 6-phosphate (Fernandes, M. J. G., Yew, S., Leclerc, D.,
Henrissat, B., Vorgias, C. E., Gravel, R. A., Hechtman, P. and Kaplan, F.
(Jan. 10, 1997), J. Biol. Chem., v. 272(2), pp. 814-820).
Acetate (Banerjee, D. K. and Basu, D., (January, 1975), Biochem. J., v.
145(1), pp. 113-118).
DMSO (dimethylsulphoxide) (Emiliani C., Falzetti, F., Orlacchio, A. and
Stirling, J. L., (Nov. 15, 1990), Biochem. J., v. 272(1), pp. 211-215).

TABLE 2

| Name | Structure | Sp. Hex. | | Hex B | Hex A |
|---|---|---|---|---|---|
| NAG-thiazoline (2.2) | 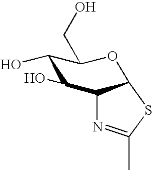 | 20 | μM⁵⁶ | 190 nM | 270 nM |
| NAGal-thiazoline (2.6) | 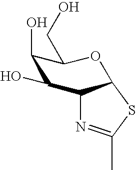 | 100 | μM⁵⁶ | 860 nM | 820 nM |
| XylNAc-isofagomine•HCI (2.7) | 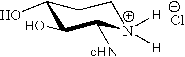 | 38 | μM | Not Done | 90 μM |

1. A method for treating an animal suffering from a disease associated with reduced activity of a lysosomal hexosaminidase by administering to the animal an effective amount of a compound which increases the activity of the hexosaminidase.

2. The method of claim 1 wherein the compound stabilises the hexosaminidase.

3. The method of claim 1 wherein the compound stabilises the alpha subunit of hexosaminidase A.

4. The method of claim 1 wherein the compound is a competitive inhibitor of hexosaminidase A.

5. The method of claim 1 wherein the disease is adult onset Tay Sachs disease, juvenile onset Tay Sachs Disease, adult Sandhoff disease or juvenile Sandhoff disease.

6. The method of claim 1 wherein the compound is a compound of the formula: 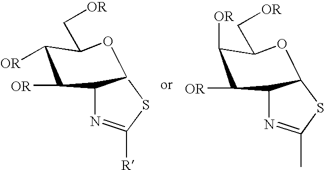
wherein R is independently selected from H, CO—CH₃, CO—Y, CO—OY and CO—NHY wherein Y is C1 to C20 alkyl; and R¹ is C1 to C20 alkyl.

7. The method of claim 6 wherein Y is C1 to C10 alkyl.

8. The method of claim 6 or 7 wherein R₁ is C1 to C10 alkyl.

9. The method of claim 6 wherein the compound is N-acetylglucosamine-thiazoline, N-acetylgalactosamine-thiazoline or an acetylated derivative thereof.

10. The method of claim 1 wherein the compound is selected from the group consisting of N-acetyl-β-D-galactosamine, 6-acetamido-6-deoxy-castanospermine, 2-acetamido-1,2-dideoxynojirimycin and 2-acetamido-2-deoxynojirimycin.

11. The method of claim 1 wherein the animal is a human.

12. The method of claim 1 wherein the animal is also treated by substrate deprivation therapy.

13. A method of modulating the activity of a mammalian hexosaminidase A enzyme comprising contacting the enzyme with a compound which stabilizes a subunit protein of the enzyme.

14. The method of claim 13 wherein the compound is selected from the group consisting of:
(a) N-acetyl-β-D-galactosamine;
(b) 6-acetamido-6-deoxy-castanospermine;
(c) 2-acetamido-1,2-dideoxynojirimycin;
(d) 2-acetamido-2-deoxynojirimycin; and
(e) a compound of the formula: 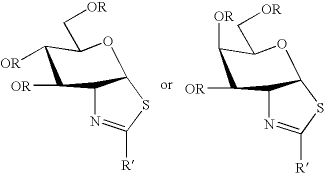
wherein R is independently selected from H, CO—CH₃, CO—Y, CO—OY and CO—NHY wherein Y is C1 to C20 alkyl; and R¹ is C1 to C20 alkyl.

15. The method of claim 13 wherein the compound is N-acetylglucosamine-thiazoline, N-acetylgalactosamine-thiazoline or an acetylated derivative thereof.

16. A method for identifying a candidate compound for treatment of a disease associated with reduced activity of a hexosaminidase comprising determining the ability of the compound to increase the activity of the hexosaminidase.

17. The method of claim 16 wherein the ability of the compound to increase heat stability of the hexosaminidase is determined.

18. The method of claim 16 wherein the ability of the compound to increase hexosamindase activity in a cell line displaying reduced hexosaminidase activity is determined.

19. A compound identified by the method of claim 16.
